(19) United States
(12) Patent Application Publication
(10) Pub. No.: US 2008/0177145 A1
(43) Pub. Date: Jul. 24, 2008

(54) Miniature fiber optic spectroscopy probes

(75) Inventor: Simon M. Furnish, New York, NY (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 12/076,581

(22) Filed: Mar. 20, 2008

Related U.S. Application Data
(62) Division of application No. 11/808,585, filed on Jun. 12, 2007.
(60) Provisional application No. 60/812,594, filed on Jun. 12, 2006.

(51) Int. Cl. A61B 1/06

(52) U.S. Cl. 600/178; 362/574

(57) ABSTRACT

The invention provides various fiber optic probe assemblies for the delivery and collection of light in small spaces. The provided probe assemblies are small, flexible and well suited to performing minimally invasive spectroscopic examinations of biological tissues in-vivo. The invention also provides intravascular catheters that include the fiber optic probe assemblies.

This application claims the benefit of U.S. provisional patent application Ser. No. 60/812,594 filed Jun. 12, 2006, which is incorporated by reference herein in its entirety.

### FIELD OF THE INVENTION

The invention relates to the fields of side-viewing fiber optic probes and catheter-based optical diagnostics.

### BACKGROUND OF INVENTION

Collection efficiency is a measure of the total light collecting capability of an optical probe. It is generally computed from the total collection fiber core area, numerical aperture (NA) and aim relative to the desired source. Numerical aperture is driven by various material constraints within the fiber and available for miniature lenses and prisms. Lens shape also effects both the direction and effective aperture for collection. For most spectroscopy applications, constraints on the miniaturization of the probe design are primarily driven by two opposing attributes: high collection efficiency and a small profile.

Existing fiber optic probes are typically found in round bundles, utilizing an “n-around-1” configuration with a central excitation fiber surrounded by an array of collection fibers. This round fiber bundle geometry then drives the selection of other components within the micro-optic assembly—the lens, filters, mirror and housing. The overall size of this probe becomes driven primarily by the size and quantity of fibers used, with a minimum diameter of 3× (3-times) the fiber diameter plus any additional packaging needs. Existing probes, such as those used for Raman spectroscopy, utilize a small spherical ball lens, roughly the same diameter as the fiber bundle diameter—which conveniently fits within thin-walled stainless steel hypodermic tubing for a robust package. Filter elements are often utilized as well to isolate the excitation signal for certain wavenumbers. For fingerprint region Raman spectroscopy, a notch filter is usually employed on the excitation fiber to remove unwanted spectral features from the excitation channel. Additional filters can also be employed at either end of the fiber to reduce the excitation signal transmitted through the collection fibers. As known in the art, the Raman fingerprint region, i.e., 200-2,500 cm⁻¹ is useful for evaluating the health of blood vessels such as arteries, including identifying and characterizing healthy tissue and atherosclerotic lesions.

Various forward and side-viewing fiber optic probe designs can be found in U.S. Pat. Nos. 6,366,726 and 5,953,477. These include various “n-around-1” designs (also known as “center-ring” designs) as well as other fiber configurations (e.g. paired fibers) utilizing shaped fiber tips, lenses and filters optimized for improved delivery and collection of light for spectroscopy. The designs described are rather complex and require expensive processing. In addition, they are still rather size limited by the particular ring-center and paired fiber configurations.

In view of the above, there is a need for a new type of side-viewing optical fiber probe that is compact, flexible and unhindered by elaborate design elements.

### SUMMARY OF INVENTION

One embodiment of the invention provides a side-viewing fiber optic probe assemblage, that includes: at least two, for example three, optical fibers arranged in a flat linear array wherein each optical fiber has a proximal end and a distal end and the elongate axes of the fibers are at least substantially parallel; and at least one beam redirecting element in optical communication with the one or more of the optical fibers at the distal end, wherein the at least one beam redirecting element is configured to direct light off-axis with respect to the fibers.

One embodiment of the invention provides a radially-viewing optical probe apparatus including two or more fiber optic probe assemblages of the invention that are radially oriented to deliver and collect light over different radial fields-of-view.

One embodiment of the invention provides a flexible intravascular optical catheter for performing spectroscopic analysis of a blood vessel wall that includes: an elongate catheter body having a proximal end and a distal insertion end; and an optical interrogation section disposed near the distal insertion end, wherein the optical interrogation section comprises at least two probe arms, each probe arm including an optical probe assemblage according to the invention disposed in a flexible tube that is radially bowed or bowable outward from the central axis of the catheter to contact or near a blood vessel wall and that is at least partly transparent so that light can be delivered and collected by the probe.

A further embodiment of the invention includes the step of optically interrogating a biological tissue, such as a blood vessel wall, using an optical probe and/or catheter according to the invention.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 shows a 4-channel, basket-type catheter of the prior art.

FIG. 2 shows the distal end basket detail of the prior art catheter design.

FIG. 3 shows the round bundle cross-section details for the prior art catheter design.

FIG. 4 shows a cross-section of the 4-channel basket catheter of the prior art within a diseased blood vessel. The targets, excitation signal and field of view for each channel are shown.

FIG. 5 shows the fiber bundle packaging details for a prior art 8-around 1 (8+1) configuration using 50/70/114 micron fibers. Smaller profile than 100 μm core fibers, but core area is reduced by 4× for the same configuration.

FIG. 6 shows a 4-around-1 (4+1) fiber bundle packaging geometry, again showing the basket section and proximal shaft section together. This configuration offers a reduced packaging size but severely reduced collection core area as well with fewer fibers. Packaging of the fibers within the proximal shaft segment is greatly simplified as the number and size of fibers is reduced, yielding a lower profile (0.83 mm).

FIG. 7 shows various fiber bundle configurations demonstrating reduction in profile and corresponding reduction in collection area. The “2+1” configuration with 100 μm core fibers is superior to the 50 μm fiber options, with a smaller profile (0.16 mm vs. 0.25 mm) for the same collection core area.

FIG. 8 shows corresponding 2+1 fiber configuration sections in a 4-channel basket catheter.

FIG. 9 shows a 2+1×100 μm core fiber basket section geometry.

FIG. 10 shows a view of the distal probe optics segment with a linear 2+1 fiber array, a flat ground spherical lens and a right angle reflector, packaged within a small U-shaped channel and transparent tubing. The lower portion of the figure shows a section view of the assembly.

FIG. 11 shows components of a 2+1 linear fiber array type core optical assembly embodiment.

FIG. 12 is a “ray trace” diagram for a single optical fiber.

FIG. 13 shows a single optical fiber with a right-angle beam redirector for side-viewing.

FIG. 14 shows a top view of the excitation and collection profile for a 3-fiber assembly embodiment.

FIG. 15 is a ray trace diagram showing a top view of a three-fiber probe embodiment with a ball lens.

FIG. 16 shows a side view of a 3-fiber probe optical assembly with either a flat ground spherical lens (radius R) or a cylindrical lens. Lenses having non-spherical shapes or curvatures may also be used.

FIG. 17 shows various single and compound lens options for a distally mounted lens. The lenses shown are ground flat to maintain the low profile of the fiber bundle. Flat lenses for use in embodiments of the invention may be formed or manufactured in any manner.

FIG. 18 shows a 3-fiber probe distal optics core provided with an optional lens on top of the bundle assembly. The optional lens is useful for shaping and directing the collection area field-of-view (FOV), but may not be suited for all situations because it takes up space within the probe profile.

FIG. 19 shows a cross-section of the “lens on top” configuration within a U-channel and assembled within a transparent outer tubing.

FIG. 20 shows a side and top view of a “lens on top” configuration.

FIG. 21 shows a front view of a “lens on top” geometry with various lens options.

FIG. 22 shows a 3-fiber array packaged in a flattened rectangular tube with an aperture cut into the top face.

FIG. 23 shows a 3-fiber array packaged on a thin, flat wire strip.

FIG. 24 shows a side and top view of a 3-fiber distal optics assembly with no filtering.

FIG. 25 shows a side and top view of a 3-fiber distal optics assembly having filters deposited directly on the fiber face.

FIG. 26 shows a side and top view of a 3-fiber distal optics assembly having a filter element placed between the fibers and lens.

FIG. 27 shows the filter element details of one embodiment. A single glass plate having three filter areas, wherein the central area has a different filter type for excitation, is shown.

FIGS. 28 shows a side and top view of a 2-fiber distal optics assembly having a filter element placed between the distal end of each fiber and the lens.

FIGS. 29A-D show various alternative lens configurations employed with 2-fiber optical probe embodiments of the invention.

FIG. 30 illustrates the cross-section of the imaging section of a basket catheter having 6 probe arms, each probe arm including a 2-fiber probe assembly according to the invention.

### DETAILED DESCRIPTION

The invention provides various fiber optic probe assemblies for the delivery and collection of light in small spaces. The provided probe assemblies are small, flexible and well suited to performing minimally invasive spectroscopic examinations of biological tissues in-vivo. The invention also provides intravascular catheters that include the fiber optic probe assemblies.

The invention is described below with reference to the appended figures.

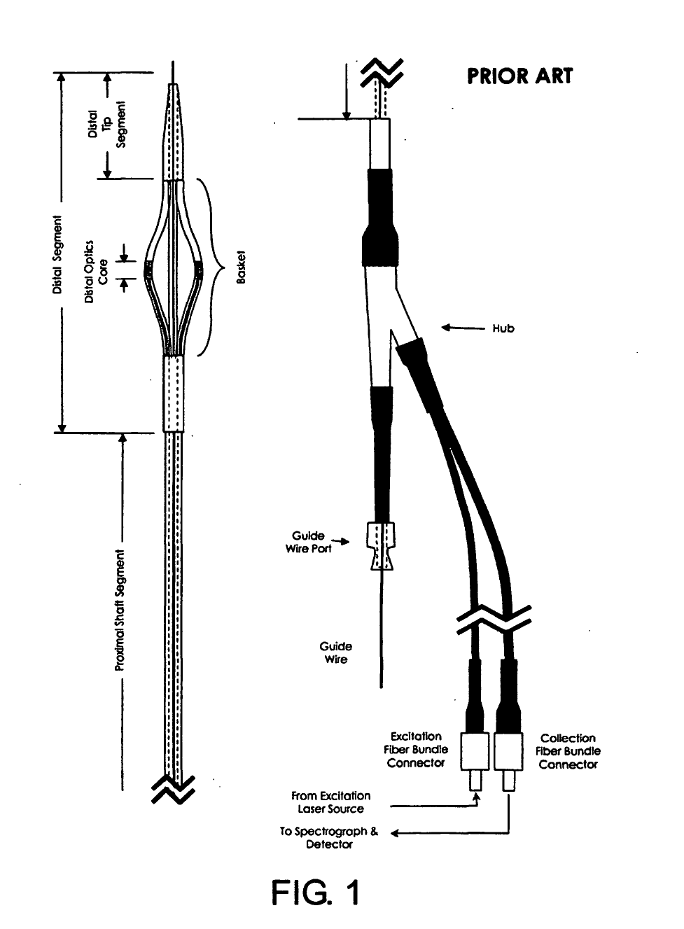

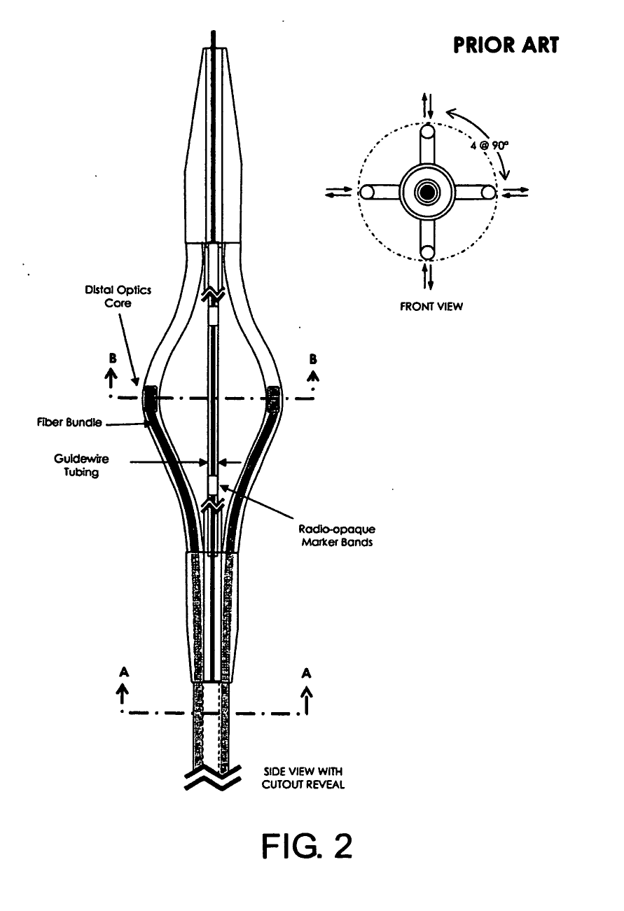

A 4-channel basket catheter profile is shown in FIGS. 1 and 2. This particular configuration is an “over the wire” catheter with a guidewire lumen passing the entire length of the catheter, and out through the “guide wire port” on the hub. The fiber bundles begin within each distal optics core and extend to the proximally to the connectors. FIG. 1 shows the proximal hub and connections used for manipulation and connecting the fibers to a laser source and spectrometer for signal analysis. FIG. 2 shows further details with respect to the indwelling end of the catheter. The side arm in this view has been partially removed to reveal the guidewire tubing. Section position A-A refers to the proximal segment section, and B-B refers to the distal basket section. A front view is provided as well and shows arrows indicating the excitation and collection of light from each channel.

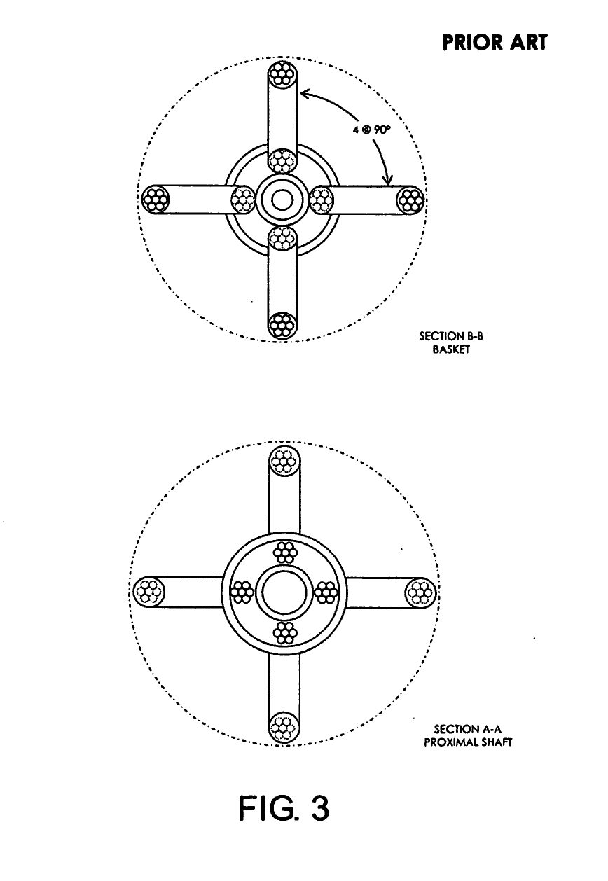

FIG. 3 shows a center-ring style bundle configuration (6-around-1) applied to the 4-channel basket profile based on Sections A-A and B-B (same positions as shown in FIG. 2). The proximal shaft profile can be improved (reduced) by fanning out the fibers and laying them in a single or dual layer around the guidewire tubing rather than keeping them in round bundles as shown.

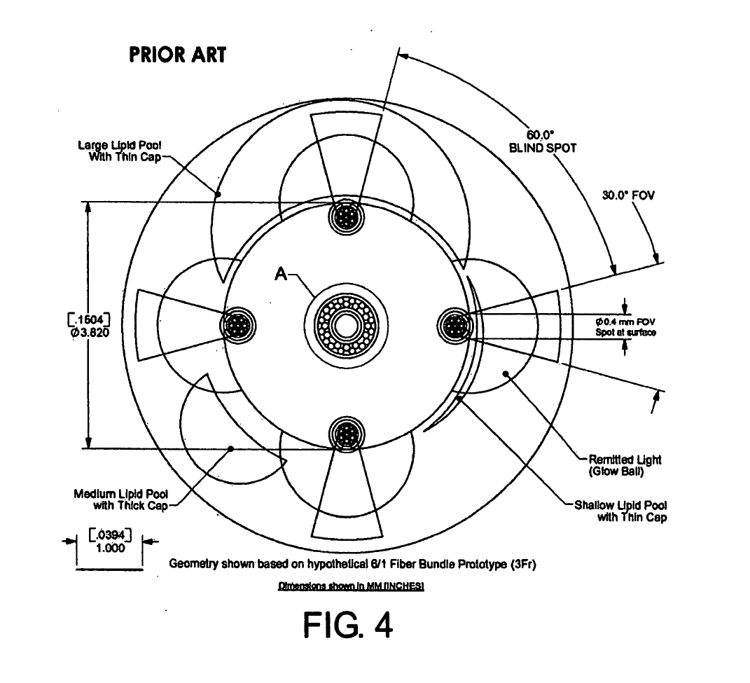

FIG. 4 shows a combined section (basket and proximal shaft) placed in an imagined blood vessel. Here, fibers in the proximal segment have been laid in a reduced profile as discussed above. FIG. 4 also shows various crescent-shaped targets representing lipid rich plaques within a blood vessel cross section. Four regions of remitted Raman light are shown as well, superimposed on the section along with a field of view for the side-viewing optics.

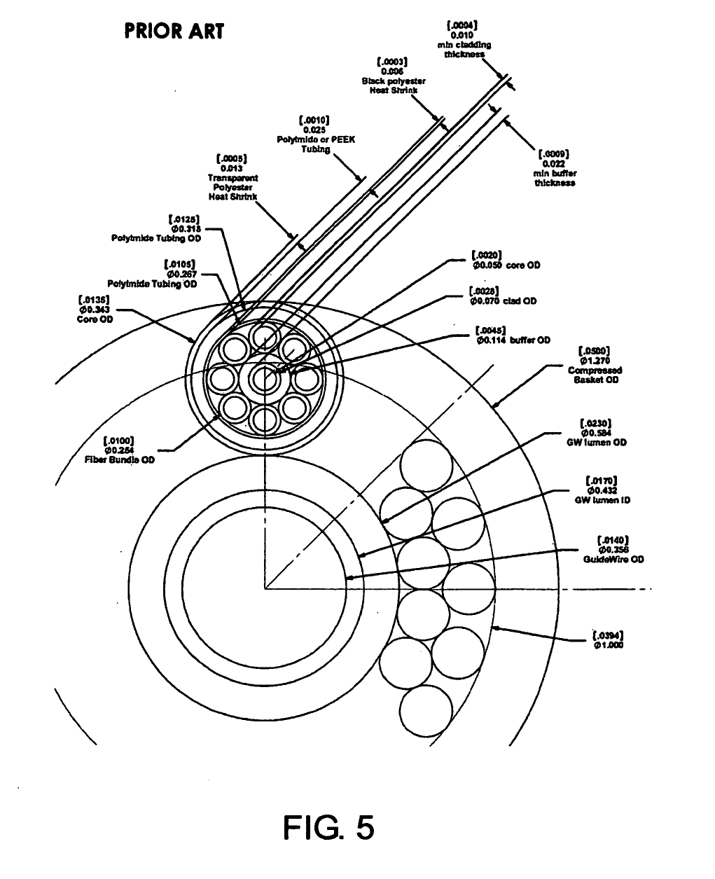

FIG. 5 shows a collapsed version of an 8+1 center-ring combination with additional packaging geometry and details. Note that, in this case, the 8+1 geometry can be achieved only by stripping the jacket from the collection fibers, allowing more collection area in the profile than 6 unstripped fibers. Using the collapsed profile of each channel, the passing profile for the catheter (the guidewire tubing outer diameter [OD] plus twice the basket arm profile diameter) can be determined. This catheter is still oversized from optimal, with the proximal shaft bundle at the 3F (French scale) limit (of usability) without a surrounding tube.

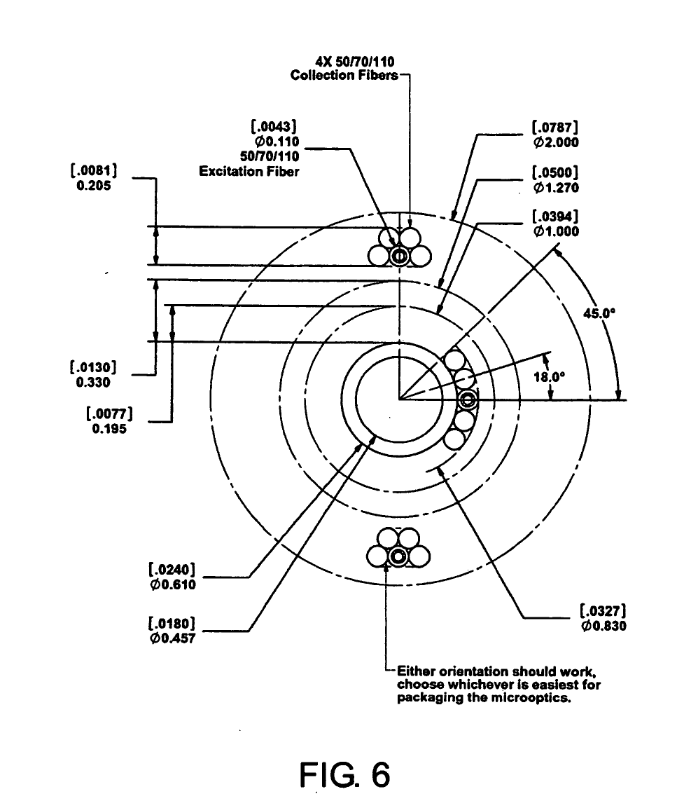

FIG. 6 shows an attempt to reduce this profile using both smaller and fewer fibers. While the geometry is better suited for a low overall profile, this configuration has half the collection core area than the 8+1 version using these same fibers, and a further fraction of the similar configuration using larger fibers.

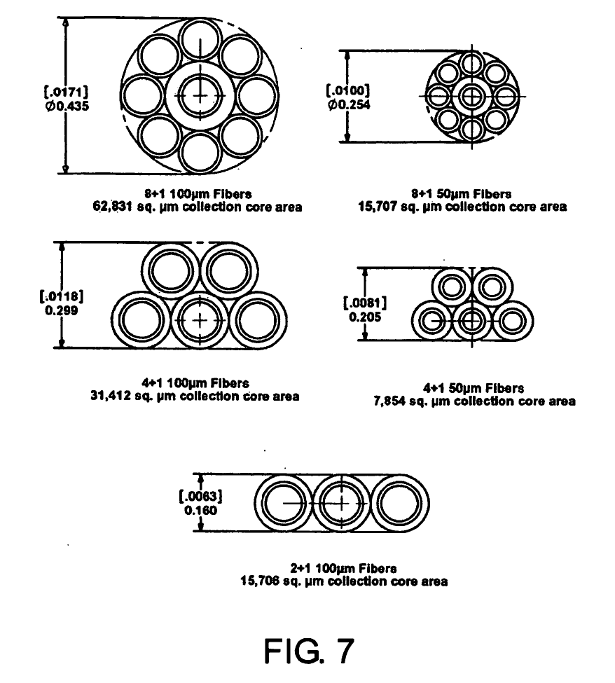

FIG. 7 shows various bundle configurations demonstrating the profile and core area based on both 100 and 50 μm core fibers. This figure also introduces a novel approach: using the 2+1 configuration shown with larger fibers yields the smallest profile without losing too much collection core area. The two other choices (4+1 and 8+1) are, by comparison, too large.

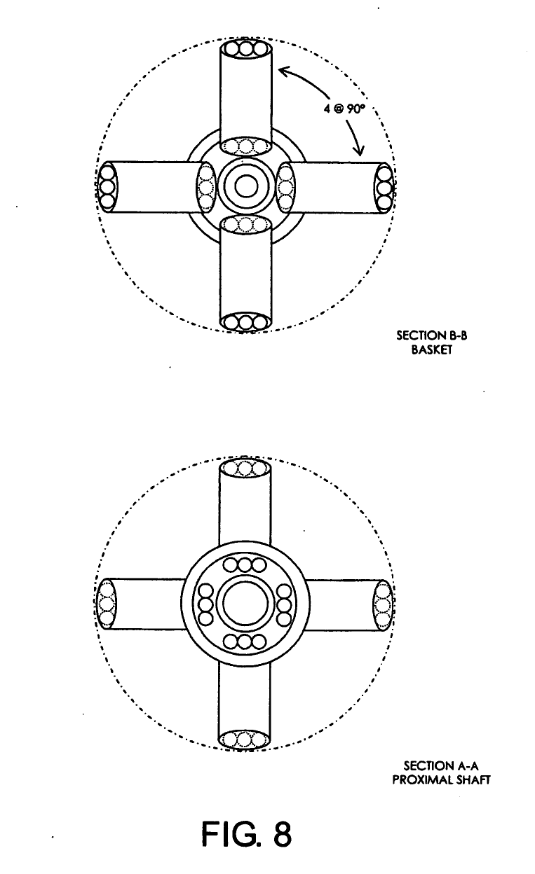

FIG. 8 shows the novel 2+1 “ribbon” configuration in the profile based on basket and proximal sections. These result in more of a flat style bundle, with improved radial flexibility due to the reduced profile.

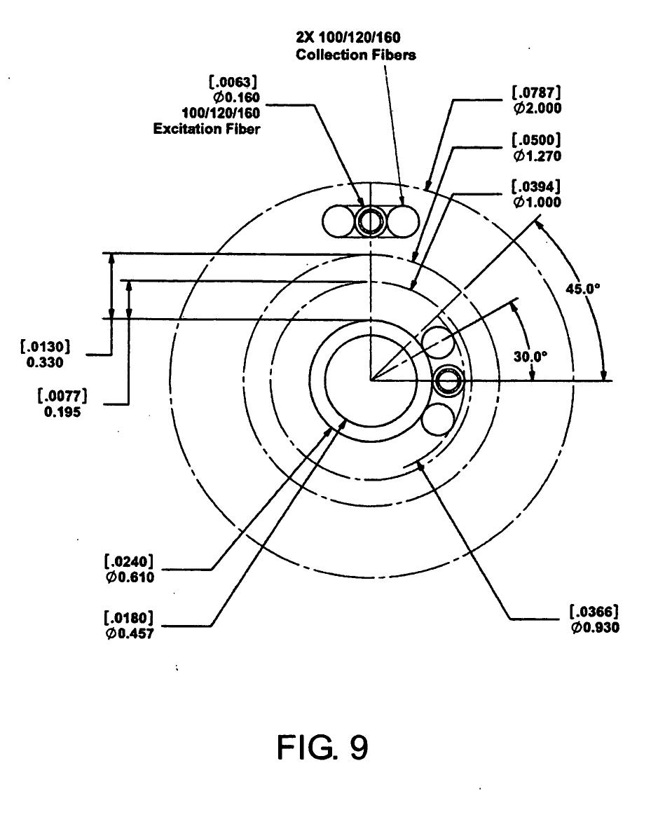

FIG. 9 shows the profile dimensions based on the novel “2+1” fiber ribbon configuration. There is plenty of room within the 1 mm/3F proximal shaft OD which will improve flexibility, deliverability and ease of manufacture. A further benefit of this configuration is the reduced cost. Fibers costs generally increase linearly with quantity, so a 12-fiber catheter (4 channels, 3 fibers per channel) should have considerably less fiber cost than a 36-fiber (4 channels, 9 fibers per channel) center ring configuration.

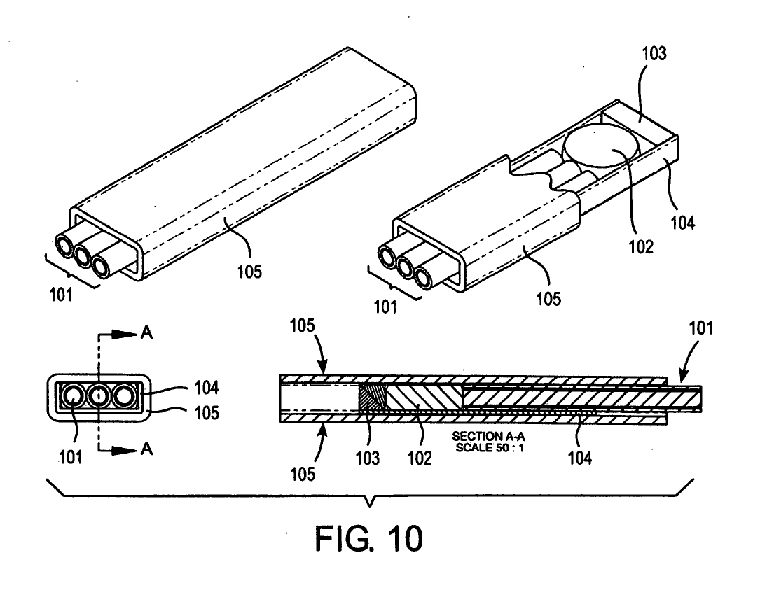

FIG. 10 shows various views of a distal probe optics segment embodiment of the invention with a linear 2+1 optical fiber array 101, a flat ground spherical lens 102 and a right angle reflector 103, packaged within a small U-shaped channel 104 and transparent tubing 105. The lower portion of the figure shows a section view of the assembly.

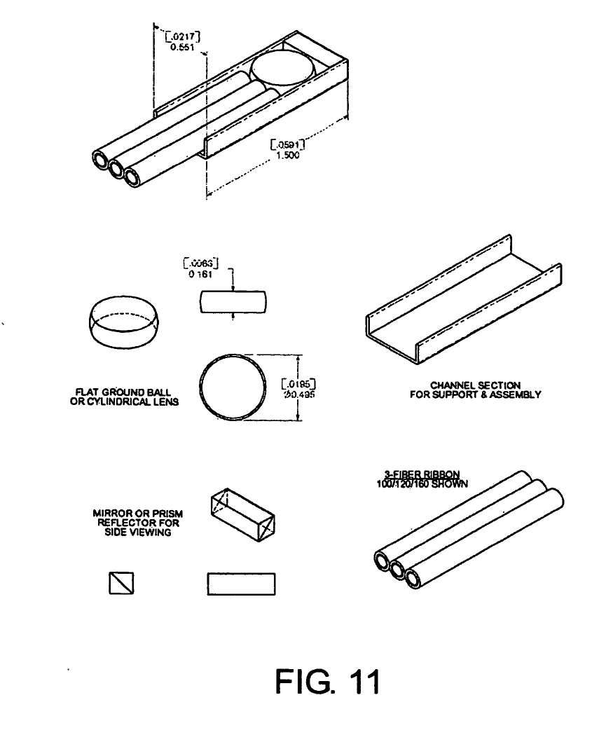

FIG. 11 shows components of the 2+1 linear fiber array type core optical assembly embodiment of FIG. 10 both assembled in the U-channel and individually (transparent tubing not shown).

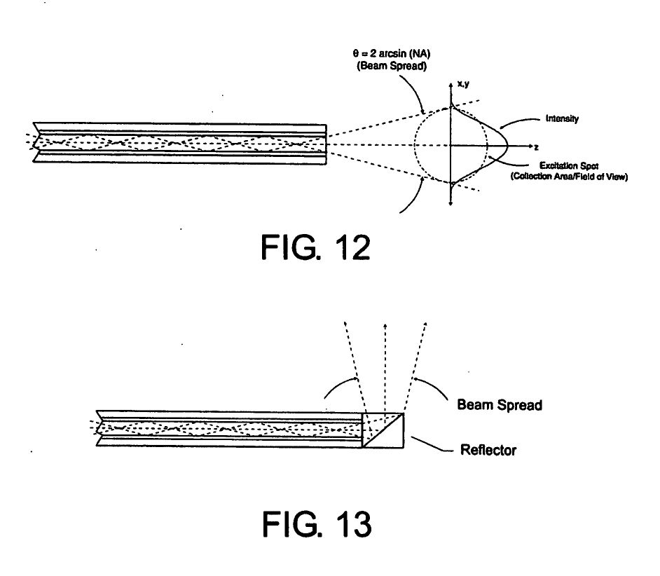

Having discussed the fiber arrangement, another important aspect of these spectroscopy probes is not just the fiber arrangement, but the entire distal core. Probe arrangements can be either forward-viewing for measuring spectra from targets directly in front of the probe, or side-viewing, for lateral views of structures not inline with the long fiber axis. The latter case is most common for inspection of the inner wall of a lumen or vessel, such as an artery or vein. Side viewing requires the redirection of light. This can be achieved in various ways, such as by use of a simple angled mirror placed in front of the fibers or an angled prism face for deflecting the excitation beam and collection areas via refraction. The mirror or prism may be a separate element positioned and held in front of the fibers, or achieved through sculpting the fibers themselves by polishing the endface of a fiber at an angle. Reflective materials such as Aluminum or Gold may be vacuum-deposited onto the reflecting surfaces to create a suitable mirror. Most-side viewing applications require roughly 90° deflection of the effective excitation beam and collection field of view. This may be achieved using a 45° right angle mirror or prism. However, deflection at angles other than 90° and beam redirectors therefor are also within the scope of the invention. FIG. 12 shows a ray tracing diagram of a single fiber. A signal intensity plot is superimposed to show where the strongest signal is transmitted through the fiber, with the full numerical aperture (NA) or beam spread limited by the extinction of this intensity on the tails of the curve. The optical fiber shown consists of a core, a cladding and an outer jacket. An “excitation spot” or “collection area” is indicated by the dashed circle for a distance away from the fiber endface. A simplified ray trace diagram of a side-viewing fiber assembly is shown in FIG. 13.

Fibers used in the probe may, for example, be selected from the broad array of fibers available and commonly used for fiber optic probes. Choice of higher NA fibers will improve the overall collection efficiency for a given type of optics. For Raman spectroscopy, low OH silica clad and core fibers are available with NA's as high as about 0.26 from suppliers such as Fiberguide Industries (Stirling, N.J. USA) and Polymicro Technologies (Phoenix, Ariz. USA). Higher NA's are achievable with other cladding materials including various polymers.

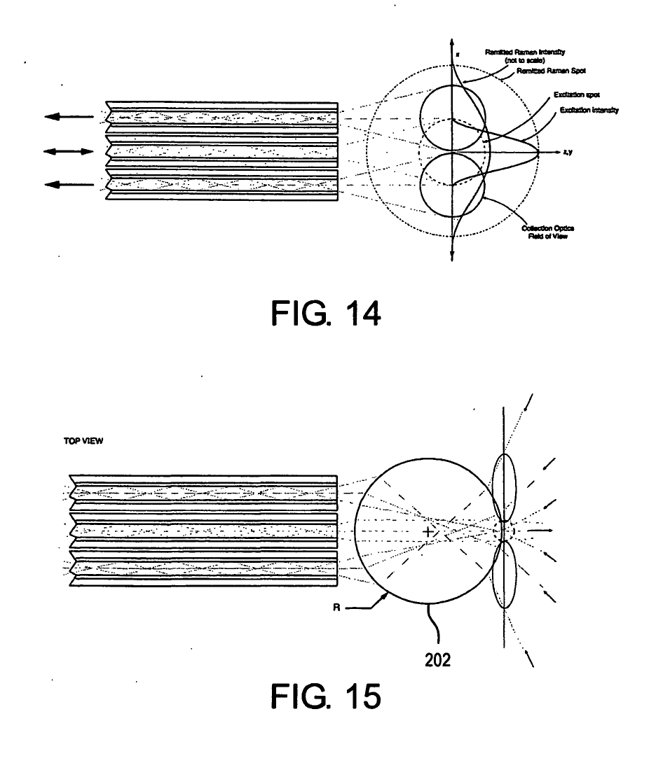

To construct a suitable distal optics core for the 2+1 linear fiber array, the various core components must be shaped in a suitable manner to align with the linear array. A simple linear array with no lenses generates an excitation and collection profile as shown in FIG. 14. The large diffuse Raman remission is shown superimposed as well. Fiber NA, lenses and reflector geometry can be modified to achieve a wide variety of excitation/collection geometries. These can be tailored for a particular function depending upon the specific application and desired collection volume.

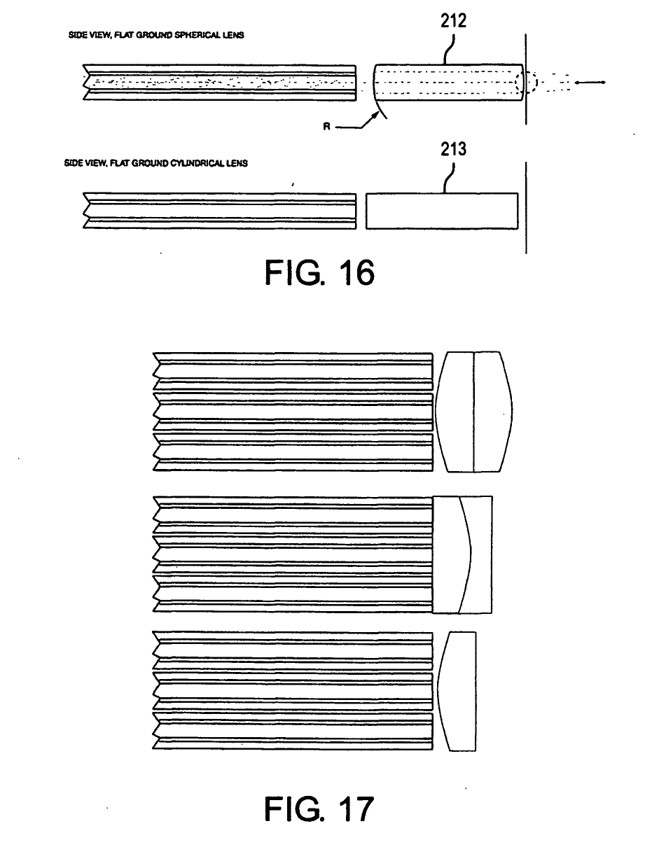

A flat linear fiber array lends itself well to the inclusion of a small, flat lens. Such a lens may be a small coin-shaped (disc-shaped) sliver cut from a polished cylindrical rod of a suitable lens material (e.g., sapphire, BK7 glass, etc.), or from polishing a ball lens on opposing sides. The cylindrical lens would result on a diverging acceptance cone stretched radially but within the plane of the flat face of the lens. FIG. 15 shows a ray-tracing diagram for the three-fiber array with a flat ball or cylindrical lens 202. A flat ground spherical ball lens would provide further curvature to expand the field of view. The lens collimates the central excitation signal while both redirecting and expanding the field of view due to the offset of the collection fibers relative to the lens center axis. The field-of-view (FOV) spots shown are not necessarily indicative of the true geometry and are for illustrative purposes only. FIG. 16 shows a side view of both a flat ground spherical lens 212 and a flat ground cylindrical lens 213 in optical communication with the fiber array of the invention. An advantage of spherical geometry is that it collimates the light and prevents it from exiting the flat face. The lenses may but do not have to be circular or spherical in shape. For example, any of the multitudes of commonly available lens shapes common to the field of optics may be used where desired. The lens may be a single or compound lens. The final choice of a particular or preferred lens shape will be based upon the balance of ease of fabrication, size constraints, overall cost and signal needs. Various examples of lens configurations are shown in FIG. 16.

A further option for lens configuration is to place the lens after the reflector in the signal path, mounted to the upper surface of the mirror-reflector assembly as shown in FIGS. 17-21. This lens takes up potentially valuable space within the profile, but also provides some potential advantages. This type of configuration is better suited for singlet style lenses with a flat face.

FIG. 17 shows various single and compound lens options for a distally mounted lens. The lenses shown are ground flat to maintain the low profile of the fiber bundle. Flat lenses for use in embodiments of the invention may be formed or manufactured in any manner. The lens and reflector prism shown in FIG. 17 are extended in size for easier fabrication and strength when bonded together with a suitable optical adhesive(s).

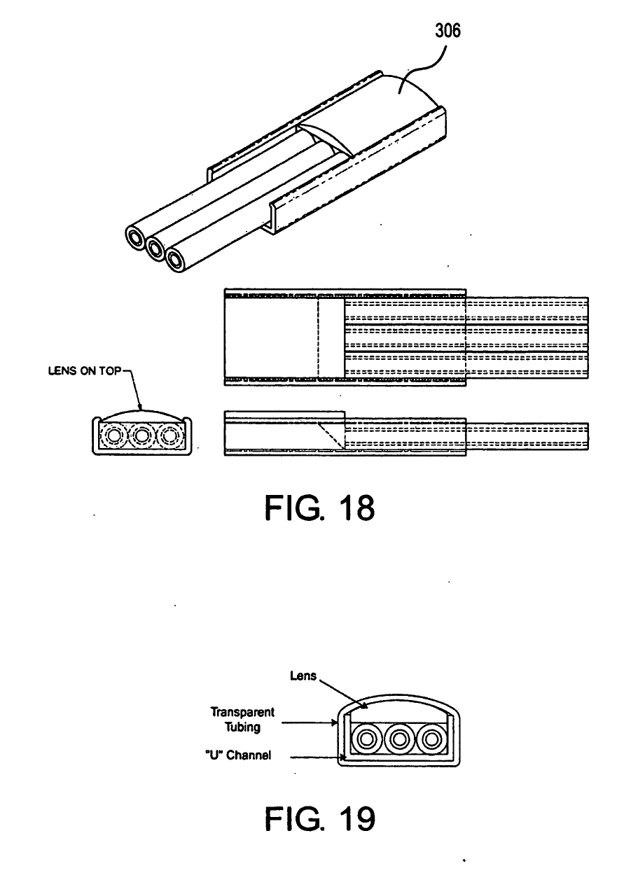

FIG. 18 shows a 3-fiber probe distal optics core embodiment provided with an optional lens 306 on top of the bundle assembly. The optional lens is useful for shaping and directing the collection area field-of-view (FOV), but may not be suited for all situations because it takes up space within the probe profile.

FIG. 19 shows a cross-section of the “lens on top” configuration mounted in a U-channel and assembled within a transparent outer tubing.

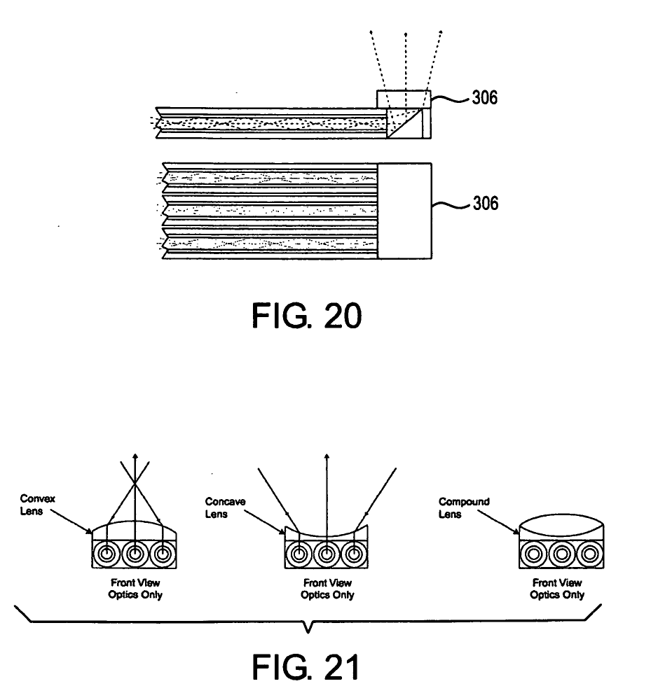

FIG. 20 shows a side and top view of a “lens on top” configuration.

FIG. 21 shows a front view of a “lens on top” geometry with various lens options.

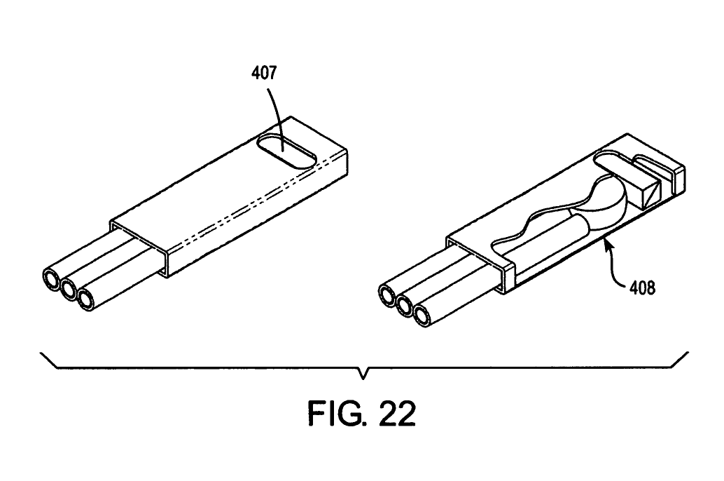

The flat-array form lends itself well to several housing options for protecting the fibers, lenses, filters and reflectors in a robust package. Several examples above have shown the small U-channel, which may, for example, be easily formed by custom extrusion, or bent from a thin sheet. The U-channel may, for example, be metallic or polymeric. Sheet metals such as stainless steel, aluminum or titanium may, for example, be used. Other possible embodiments include an extruded rectangular-shaped tube with a window or aperture 407 provided (e.g., cut or punched) from above as shown in solid and cut-away views in FIG. 22. A backing plate or strip 408 is joined to the U-channel as shown forming a rectangular housing for the optical components.

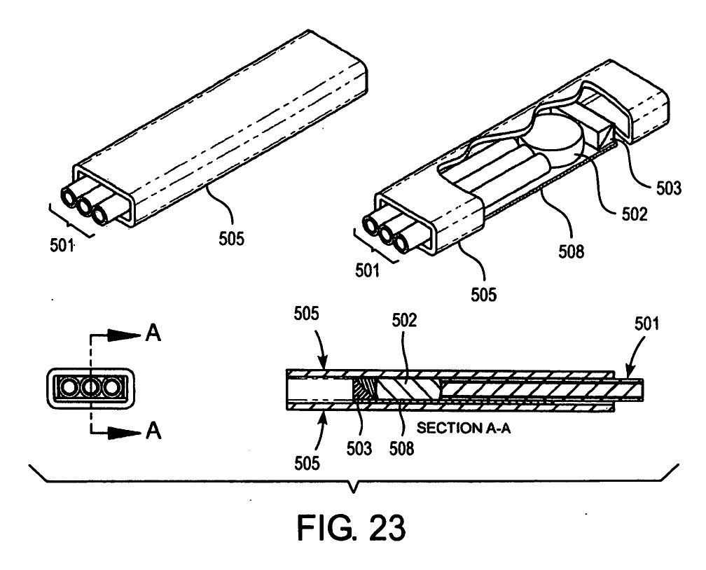

FIG. 23 shows a further embodiment, with a single flat strip 508 to provide a backbone for the fiber and lens assembly. Here, a U-channel is not provided. Instead the optical components are secured in position to strip 508 and the optical assemblage is disposed in a suitably transparent tubular enclosure 505. In this embodiment, securing the optical components in position is more reliant on the bonding the elements together (for example with epoxy adhesive) than on physical constraints of a U-channel or tubular housing of other embodiments. In any embodiments of the probe assemblages, apparatuses and catheters of the invention, the optical probe assemblage(s) may be housed in an at least substantially rectangular or “ribbon-shaped” housing structure. The edges of such a rectangular structure may be rounded-off or otherwise smoothed.

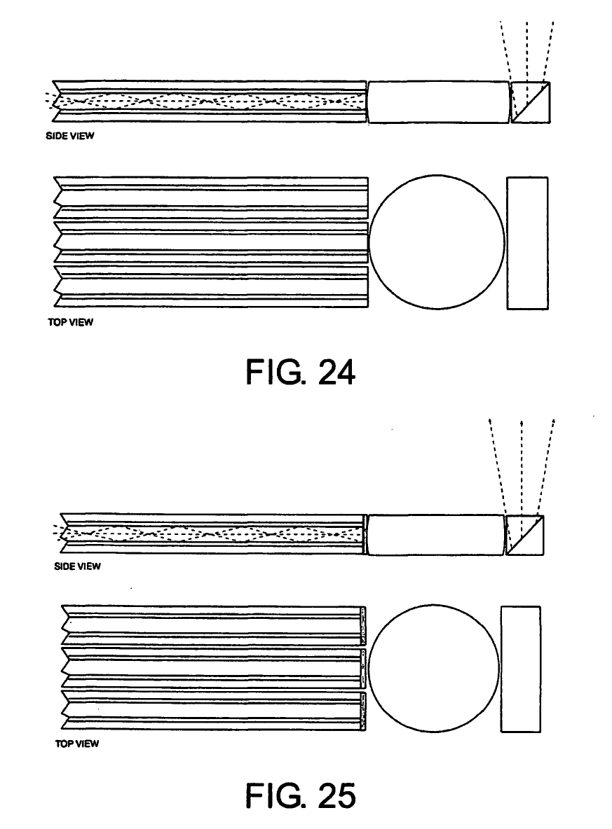

Filtering is an option that is desirable for certain applications. For example, fiber fluorescence background is problematic for fingerprint region Raman measurements. In this case, a filter is desirable on the excitation side to confine the excitation wavelength and block any other wavelengths that may have been generated by the excitation light traveling down the fiber. A second and type of filter is needed on the collection side to reduce excitation light from being collected, but should allow the desired Raman bands to pass undisturbed. Optical filters are commonly available through companies such as Barr Associates (Westford, Mass. USA) and others. There are several approaches to applying filters to the 3-fiber array. FIG. 24 shows a top and side view of the general side-viewing 3-fiber distal optics with a lens and no filtering. FIG. 25 shows a preferred embodiment with filters 609 applied directly to the face of the fibers. Using this technique, filters can be applied to large batches of fibers at once and no additional parts are required. For fingerprint region Raman spectroscopy applications, the central excitation fiber may be provided with a filter that passes an extremely narrow excitation bandwidth, rejecting any additional wavelengths generated such as the Raman-background contribution of the fiber itself (silica-scattered light). Each of the surrounding collection fibers may be provided with a notch-filter to reject the excitation band but pass Raman signal (in the fingerprint region) generated by the target.

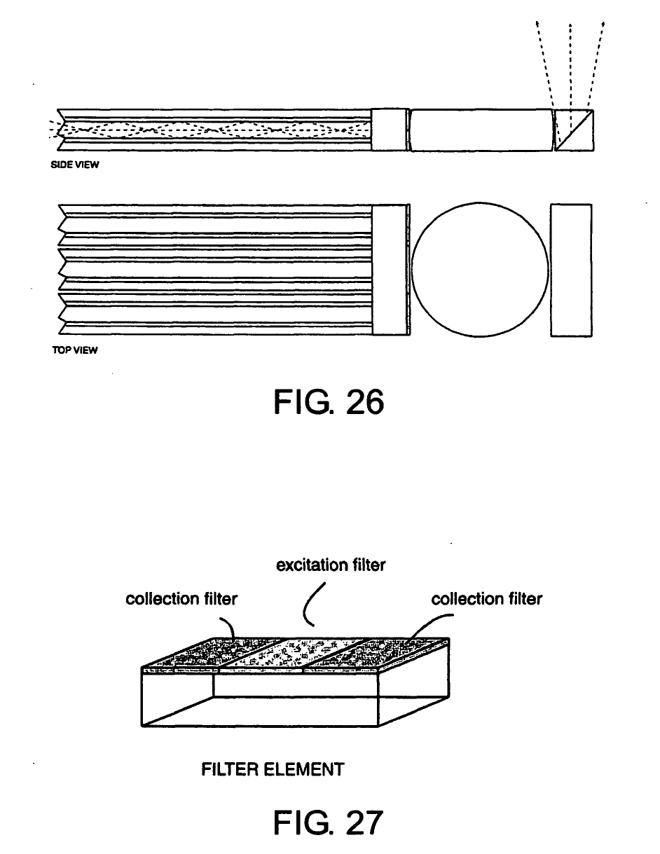

FIG. 26 shows another option, with a filter 707 element placed in the optical path between fibers and the lens. The filter element has three different filter areas created through a process of photo-masking to apply different coating types to different areas of the filter as shown in FIG. 27. This type of filter is practical because a long strip of filters can be made and then diced into individual elements. Filters may also be integrated within the optical fiber(s).

### EXAMPLE OF A PREFERRED EMBODIMENT

In a preferred embodiment, a fiber optic probe is constructed from three identical 2 m long 0.26-NA, low-OH silica core and clad optical fibers having an acrylate jacket. The fibers have a 0.10 mm core diameter, 0.01 mm thick cladding and a 0.02 mm thick jacket for a total fiber diameter of 0.160 mm. The silica indices of refraction are selected to have a 0.26 NA, yielding a 30° beam spread or acceptance angle. At the proximal end, each fiber has a separate small form factor LC-style fiber connector. At the distal end, the fibers have a polished face and are placed within a small channel made from thin stainless steel. A spherical sapphire ball lens with a diameter of 0.48 mm that has been ground flat is placed just in front of the polished fiber faces. On the side opposite the fibers, a right angle mirror is positioned to reflect the light off axis. All of these components are fixed in position relative to one another using an optical grade epoxy. The fiber bundle and distal core are placed within a thin transparent plastic tube to protect the optics from the surrounding environment. The central fiber is connected to a frequency stabilized laser source and the outer two fibers are connected to a spectrograph with appropriate filtering for analysis of Raman scattered light.

In an alternate embodiment, the structural U-channel element is replaced with a thin flat-wire strip (resembling the U-channel, but without the sides). Maintaining the flat ribbon alignment of the fibers may is more difficult without the sidewall structures, and such an embodiment will be more dependent upon the adhesive bonds between the fibers, optical elements and strip therein.

In an alternate embodiment, there is no underlying structural component, and the fibers and micro-optics are affixed together using an optical grade epoxy adhesive, then compressed within a thin heat-shrink tube. The invention is not limited to the manner by which the fibers and micro-optical components are secured in place and/or to each other. Indeed, many different ways may be apparent to those skilled in the art. As used herein, the term “tube” should be construed broadly as including tubular structures, partially tubular structures (lumen not extending entire length of structure) and rods in which the optical probes of the invention may be disposed.

In a further embodiment, four or more identical 3-fiber probes are placed within a single intravascular catheter for optically interrogating a tissue, for example a blood vessel wall such as that of an artery or vein. For example, Raman spectroscopic diagnosis of an arterial wall may be performed. In this embodiment, each probe exists as a separate channel within the catheter for viewing in a discrete radial direction. Examples of a four-channel basket are shown in FIGS. 1 and 2 and six-channel basket designs are shown in FIGS. 8 and 9.

In a further embodiment, the fiber connectors are combined into a single connector with multiple ferrules for each fiber.

In a further embodiment, the excitation fiber connectors are combined into a single connector, and the collection fibers are bundled together into a separate connector.

A multitude of fiber connectors are commercially available, many of which are suitable for this application in a variety of combinations.

Two-fiber probe embodiments are also provided by the invention.

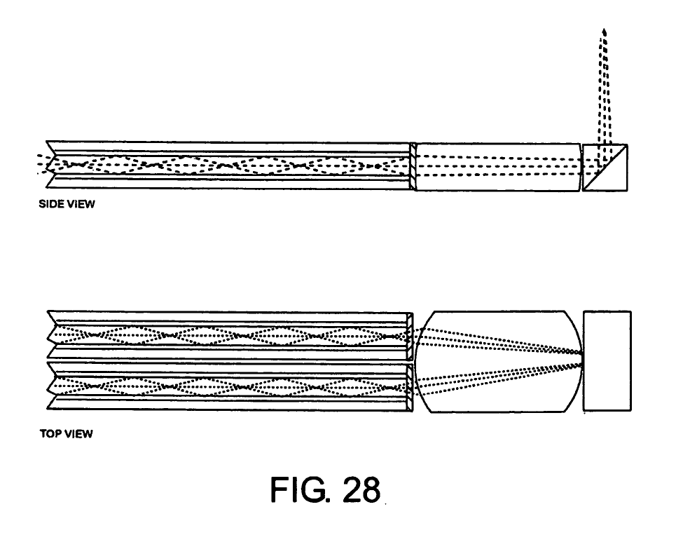

FIG. 28 shows a top and side view of 2-fiber probe embodiment having an excitation fiber and a collection fiber, each with a separate filter at the end. For fingerprint region Raman spectroscopy, the light emerges from excitation fiber, filtered to provide an extremely narrow excitation bandwidth, cutting out any additional wavelengths generated such as Raman contribution of the fiber itself. The excitation light is then focused and directed to a point further downstream, ideally to coincide with the collection fiber's field of view at the target surface. The lens geometry and location relative to each fiber dictates the beam output pattern in terms of focus and beam redirection (aim). The lens in this case is a truncated sphere—ground flat on the top and bottom (relative to the side view) into a coin-shaped (disc-shaped) lens, and ground further on each sides to the width of the two fibers for a reduced total optical package size. The rectangular component distal to this lens is a mirror element for redirecting the light out of the plane of the two fibers and into the tissue, at an angle of approximately 10 to 90 degrees from the horizontal. A 45-degree mirror is shown, which redirects the light 90 degrees from the horizontal. A portion of the light scattered by the target is collected by traveling in the reverse trajectory into either fiber. A notch-filter on the collection fiber rejects the excitation band but collects and passes the Raman signal generated by the target (in the fingerprint region). Since the geometry is symmetric, the choice of excitation and collection fiber location simply depends upon the positioning of each filter. The filters may be a separate component—deposited on a thin sliver of transparent material, or optionally deposited directly on the end of the fiber or integrated into a fiber.

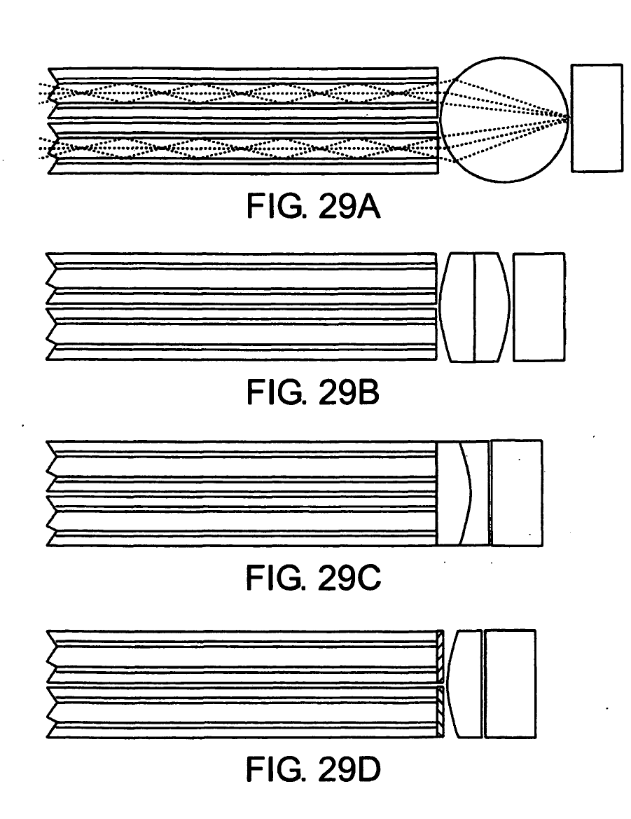

FIG. 29 shows various lens options in top view for 2-fiber probe embodiments, all of which would have a similar flat appearance in the side view as shown in FIG. 28. The lenses include a smaller ball-lens or cylindrical lens, and various combinations of compound and singlet lenses for achieving the desired output. The filters are not shown in some of these views for simplicity and represent an embodiment with the filters directly deposited onto the fiber face.

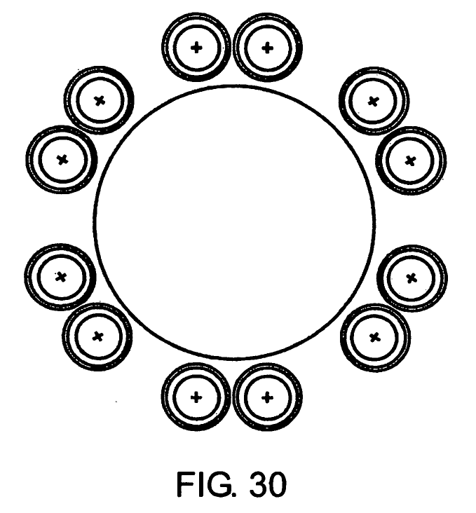

FIG. 30 shows a cross section view of the dual fiber arrangement in a basket-type catheter with 6 “scanning arms” and show how a smaller number of fibers per scanning arm allows for an increased number of scanning arms using the same number of fibers. In this example, a 6×2-fiber basket configuration increases the radial resolution for the catheter by 50% in comparison to a 4×3-fiber configuration.

In addition to Raman spectroscopy, the optical assemblies and catheters of the invention may be used to implement a variety of optical diagnostic techniques such as, but not limited to, fluorescence spectroscopy, time-resolved fluorescence spectroscopy (e.g., laser-induced), laser speckle spectroscopy and imaging, NIR spectroscopy and optical coherence tomography (OCT). The optical assemblies and catheter of the invention may also be used to deliver a therapeutic amount of light to a target tissue diagnosed by the apparatus, such a diseased section of a blood vessel. The therapeutic light delivery may, for example, be part of Photodynamic Therapy (PDT), i.e., involve photoactivation of a photosensitizer agent or it may involve light treatment alone, e.g., irradiation with infrared light. Accordingly, one embodiment of the invention provides a method for inspecting a blood vessel such as an artery or vein that includes the step of optically interrogating at least a portion of the blood vessel using any of the optical assembly and/or catheters of the invention described herein. A related embodiment includes the steps of optically interrogating at least a portion of the blood vessel such as an artery or vein, using any of the optical assembly and/or catheters of the invention described herein and delivering a therapeutic amount of light using the optical assembly and/or catheter to at least a part of the blood vessel that is diagnosed from the interrogation as needing treatment with the therapeutic amount of light.

The optical assemblies and catheters of the invention described herein are well suited to the detection, location and/or characterization of atherosclerotic plaques and, in particular, vulnerable plaques. Recent clinical data suggests that the majority of heart attacks result from the rupture of vulnerable plaques rather than hard calcified plaques. In many instances, vulnerable plaques do not impinge on the vessel lumen but, instead, are embedded in the wall of an artery. Effective detection and treatment of vulnerable plaques is complicated since a patient typically does not experience angina and since conventional angiography or fluoroscopy techniques are not well suited for detecting such plaques. Vulnerable plaques have characteristic physical, chemical and biological signatures. The majority of vulnerable plaques include a lipid pool, a necrotic ring, and a dense infiltrate of macrophages contained by a thin fibrous cap, generally having a thickness of 50 or fewer microns (micrometers). Some fibrous caps may even have a thickness of around 2 microns

The following patents and publications teach methods that may be used for detecting, locating and/or characterizing vulnerable plaques and which may, for example, be employed using the side-viewing optical assemblies and catheters of the present invention. Raman spectroscopy-based methods and systems are disclosed, for example, in: U.S. Pat. Nos. 5,293,872; 6,208,887; and 6,690,966; and in U.S. Publication No. 2004/0073120, each of which is incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is incorporated by reference herein in its entirety. U.S. Publication No. 2002/0071474, each of which is incorporated herein in its entirety. Time-resolved laser-induced fluorescence spectroscopy (TR-LIFS) may also be used to detect and locate vulnerable plaques. U.S. Pat. No. 6,272,376 teaches TR-LIFS methods for detecting lipid-rich vascular lesions and is incorporated by reference herein in its entirety.

Each of the patents and other publications cited in this disclosure is hereby incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

1.-18. (canceled)

19. A flexible intravascular optical catheter for performing spectroscopic analysis of a blood vessel wall:
an elongate catheter body having a proximal end and a distal insertion end;
an optical interrogation section disposed near the distal insertion end, wherein the optical interrogation section comprises at least two probe arms, each probe arm comprising:
a fiber optic probe assemblage according to claim 1 disposed in a flexible tube that is radially bowed or bowable outward to contact or near a blood vessel wall and that is at least partly transparent so that light can be delivered and collected by the probe.

20. The catheter of claim 19, wherein each probe arm has a soft rounded-off profile to minimize trauma to a blood vessel wall.

21. The catheter of claim 19, wherein each probe arm exerts a slight outward radial force to promote contact with the lumen wall.

22. The catheter of claim 20, wherein each probe arm exerts a slight outward radial force to maintain contact with the lumen wall.

23. The catheter of claim 19, further configured with a guidewire lumen passing either a partial length or the entire length of the catheter.

24. The catheter of claim 19, having four separate probe arms at least substantially evenly spaced at 90.degree. for performing four-quadrant measurements.

25. The catheter of claim 20, having four separate probe arms at least substantially evenly spaced at 90.degree. for performing four-quadrant measurements.

26. The catheter of claim 19, comprising a plurality of probe arms wherein the probe arms are at least substantially equally radially spaced.

27. (canceled)

28. (canceled)

29. A flexible intravascular optical catheter for performing spectroscopic analysis of a blood vessel wall comprising:
an elongate catheter body having a proximal end and a distal insertion end;
an optical interrogation section disposed near the distal insertion end, wherein the optical interrogation section comprises at least two probe arms, each probe arm comprising:
a side-viewing fiber optic probe assemblage comprising: at least two optical fibers arranged in a flat linear array wherein each optical fiber has a proximal end and a distal end and the elongate axes of the fibers are at least substantially parallel; and at least one beam redirecting element in optical communication with one or more of the optical fibers at the distal end, wherein the at least one beam redirecting element is configured to direct light off-axis with respect to the fibers, wherein the side-viewing fiber optic probe assemblage is disposed in a flexible tube that is radially bowed or bowable outward to contact or near a blood vessel wall and that is at least partly transparent so that light can be delivered and collected by the probe.
